(19) United States
(12) Patent Application Publication
(10) Pub. No.: US 2025/0316392 A1
(43) Pub. Date: Oct. 9, 2025

(54) AI BASED SYSTEM AND METHOD FOR PERSONALIZED HEALTH DATA MANAGEMENT

(71) Applicant: Cognizant Technology Solutions US Corp, Teaneck, NJ (US)

(72) Inventors: Gauravkumar Brijmohandas GANDHI, Auckland (NZ); Altaf Parkar, Auckland (NZ); Suresh Kadiyam, Auckland CBD (NZ); Kishan Gunaratna, Auckland (NZ); Girish Sivakumar, Victoria (AU)

(21) Appl. No.: 19/169,825

(22) Filed: Apr. 3, 2025

(30) Foreign Application Priority Data
Apr. 5, 2024 (NZ) 809888

(51) Int. Cl. G16H 50/30; G06F 16/34; G06F 40/295; G06V 30/10; G16H 10/60

(52) U.S. Cl. CPC G16H 50/30; G06F 16/345; G06F 40/295; G06V 30/10; G16H 10/60

(57) ABSTRACT

An AI based system and a method for personalized health data management is provided. The invention provides for performing one or more data extraction operations on one or more data types to obtain processed data types. The data types are collected from multiple data sources. The processed data types are analyzed for detecting abnormalities and deviations in the collected data types by providing a sequence of prompts to AI models. One or more health features data is extracted from the analyzed data types by using feature extraction techniques. Machine learning models are employed to augment the extracted health features data in order to identify anomalies and patterns in the health features data. Insights and recommendations associated with health of a user are generated based on processing of the analyzed features data. Action items are triggered based on the generated insights and recommendations.

This application claims the benefit of New Zealand Provisional Application No. 809888 dated Apr. 5, 2024, which is incorporated by reference in its entirety.

### FIELD

This technology relates generally to the field of health data management. More particularly, this technology relates to an artificial intelligence-based system and a method for personalized health data management.

### BACKGROUND

Health data management has become a prerequisite in this fast moving and busy landscape for tracking health related data and providing recommendations for health improvement. Generally, health data tracking is done by static processes which are often unable to process and adapt to the vast and continuously growing volumes of health data, thereby reducing effectiveness in providing accurate and timely health recommendations. Also, health data tracking sometimes requires manual intervention which further restricts scalability and makes it challenging to manage health data of a large population efficiently. Also, static processes and manual interventions lack the advanced predictive capabilities required to anticipate future health events and trends. This shortfall limits the ability to proactively manage health conditions and prevent potential health issues before they arise. It has been observed that current health data management systems are not capable of adapting in real-time to users' changing health needs and preferences.

Further, it has been observed that the traditional health data management systems often struggle with integrating data from various sources such as electronic health records (EHRs), wearable devices, and patient-reported outcomes. This fragmentation leads to incomplete health profiles and hinders the ability to provide comprehensive and personalized health recommendations. Furthermore, current systems often fail to effectively engage with finance and insurance organizations, thereby resulting in a disjointed experience for patients and customers. This inefficiency often leads to delays in claims processing, coverage approvals, and overall dissatisfaction with the healthcare experience.

In light of the aforementioned drawbacks, there is a need for a system and a method which provides for efficient personalized health data management. There is a need for a system and a method which provides for effectively processing a huge volume of health data. Further, there is a need for a system and a method which provides for eliminating or reducing manual intervention in health data management. Furthermore, there is a need for a system and a method which provides for real-time adaptation of health management systems to changing health needs and preferences.

### SUMMARY

In various embodiments of the present invention, an Artificial Intelligence (AI) based system for personalized health data management is provided. The system comprises a memory storing program instructions, a processor executing instructions stored in the memory, and a health data management engine executed by the processor. The health data management engine performs one or more data extraction operations on one or more pre-processed data types to obtain processed data types. The data types are collected from multiple data sources. The health data management engine analyzes the processed data types for detecting abnormalities and deviations in the collected data types by providing a sequence of prompts to AI models. The health data management engine extracts one or more health features data from the analyzed data types by using one or more feature extraction techniques. The health data management engine employs one or more machine learning models to augment the extracted health features data in order to identify anomalies and patterns in the health features data. The health data management engine generates insights and recommendations associated with health of a user based on processing of the analyzed features data. The one or more action items are triggered based on the generated insights and recommendations.

In various embodiments of the present invention, an AI based method for personalized health data management is provided. The method is implemented by a processor executing instructions stored in a memory. The method comprises performing one or more data extraction operations on one or more data types to obtain processed data types. The data types are collected from multiple data sources. The method comprises analyzing the data types for detecting abnormalities and deviations in the collected data types by providing a sequence of prompts to AI models. The method comprises extracting one or more health features data from the analyzed data types by using one or more feature extraction techniques. The method comprises employing one or more machine learning models to augment the extracted health features data in order to identify anomalies and patterns in the health features data. The method comprises generating insights and recommendations associated with health of a user based on processing of the analyzed health features data. One or more action items are triggered based on the generated insights and recommendations.

In various embodiments of the present invention, a computer program product is provided. The computer program product comprises a non-transitory computer-readable medium having computer-readable program code stored thereon, the computer-readable program code comprising instructions that, when executed by a processor, cause the processor to perform one or more data extraction operations on one or more data types to obtain processed data types. The data types are collected from multiple data sources. The processed data types are analyzed for detecting abnormalities and deviations in the collected data types by providing a sequence of prompts to AI models. One or more health features data is extracted from the analyzed data types by using one or more feature extraction techniques. One or more machine learning models are employed to augment the extracted health features data in order to identify anomalies and patterns in the health features data. Insights and recommendations associated with the health of a user are generated based on processing of the analyzed features data. One or more action items are triggered based on the insights generated and recommendations.

### BRIEF DESCRIPTION OF THE ACCOMPANYING DRAWINGS

Examples of the present invention are described by way of embodiments illustrated in the accompanying drawings wherein:

FIG. 1 is a detailed block diagram of an artificial intelligence-based system for personalized health data management, in accordance with an embodiment of the present invention;

FIG. 2 and FIG. 2A illustrate a flowchart depicting an artificial intelligence-based method for personalized health data management, in accordance with an embodiment of the present invention; and

FIG. 3 illustrates an exemplary computer system in which various embodiments of the present invention may be implemented.

### DETAILED DESCRIPTION

Examples of the present invention disclose an Artificial Intelligence (AI) based system and a method which provides for personalized health data management in an automated manner. Examples of the present invention provide for a system and a method for effectively processing a large volume of health data for generating healthcare related insights and recommendations. Further, Examples of the present invention provide for a system and a method for eliminating or reducing manual intervention in health data management for fast and accurate health data management. Also, Examples of the present invention provide for a system and a method for real-time adaptation of health management systems to changing health needs and preferences. Furthermore, Examples of the present invention provide for a system and a method for integrating data from various sources such as electronic health records (EHRs), wearable devices, and patient-reported outcomes. Yet further, Examples of the present invention provide for a system and a method for proactively engaging with finance and insurance organizations, for enhancing experience for patients and customers.

The disclosure is provided in order to enable a person having ordinary skill in the art to practice the invention. Exemplary embodiments herein are provided only for illustrative purposes and various modifications will be readily apparent to persons skilled in the art. The general principles defined herein may be applied to other embodiments and applications without departing from the scope of the invention. The terminology and phraseology used herein is for the purpose of describing exemplary embodiments and should not be considered limiting. Thus, the present invention is to be accorded the widest scope encompassing numerous alternatives, modifications, and equivalents consistent with the principles and features disclosed herein. For purposes of clarity, details relating to technical material that is known in the technical fields related to the invention have been briefly described or omitted so as not to unnecessarily obscure the present invention.

Examples of the present invention would now be discussed in context of embodiments as illustrated in the accompanying drawings.

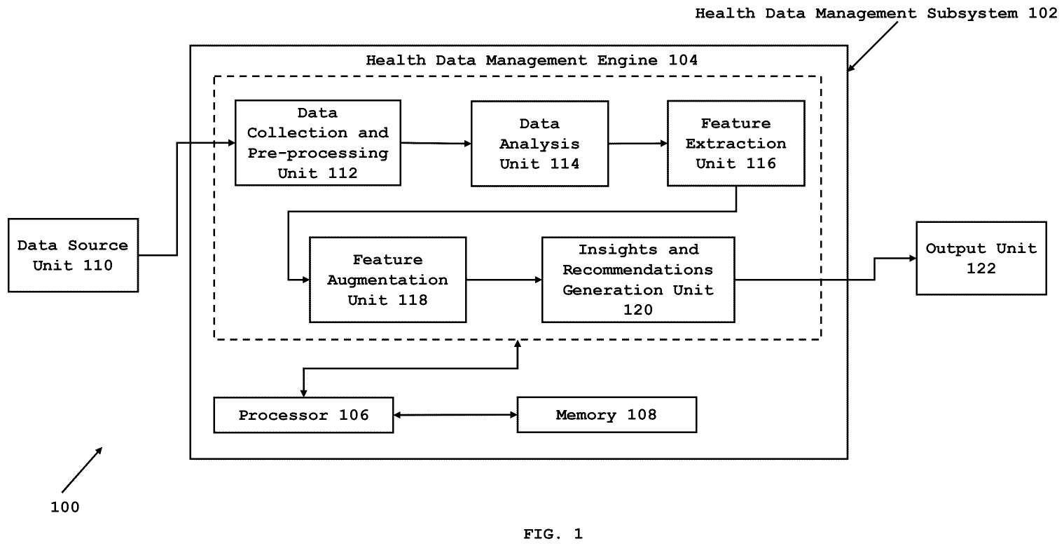

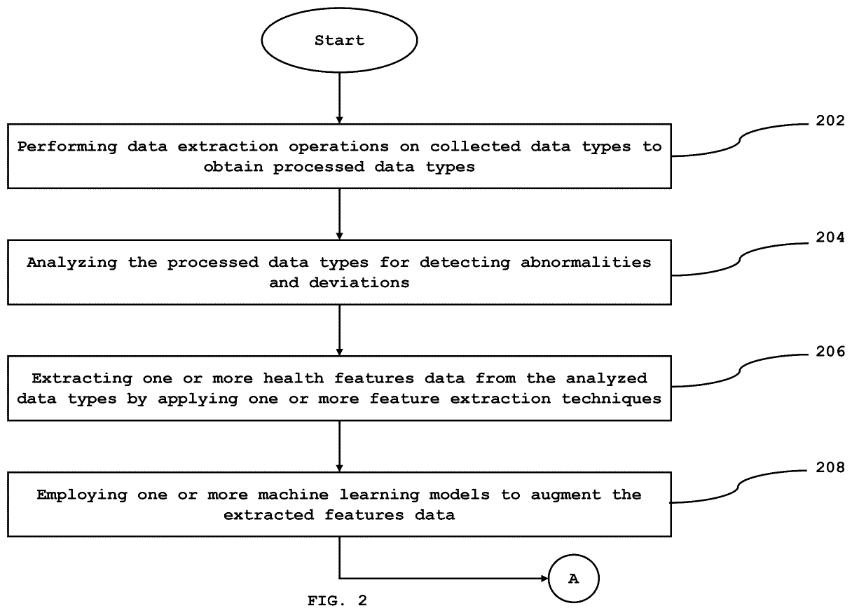

FIG. 1 is a detailed block diagram of an AI based system 100 for personalized health data management, in accordance with various embodiments of the present invention. Referring to FIG. 1, in an embodiment of the present invention, the system 100 comprises a health data management subsystem 102 (subsystem 102), a data source unit 110 and an output unit 122. The data source unit 110 and the output unit 122 are connected to the subsystem 102 via a communication channel (not shown). The communication channel (not shown) may include, but is not limited to, a physical transmission medium, such as, a wire, or a logical connection over a multiplexed medium, such as, a radio channel in telecommunications and computer networking. Examples of radio channel in telecommunications and computer networking may include, but are not limited to, a local area network (LAN), a metropolitan area network (MAN) and a wide area network (WAN).

In an embodiment of the present invention, the subsystem 102 is configured with Gen AI agents for providing real-time personalized health data management in an automated manner. The subsystem 102 captures comprehensive health data from various sources and wearable gadgets and analyzes the data to provide emergency services and user specific suggestions for medications, supplements, and nutritious meals. Further, the subsystem 102 tracks the health data of a user for providing insights and personalized health recommendations. The subsystem 102 has an in-built functionality which enables health data analysis based on a user request. Further, the subsystem 102 leverages GenAI agents to analyze the data from health documents or from other sources, processes the health situation and triggers an action item such as an emergency service, a collaboration service or a recommendation service.

In an embodiment of the present invention, the subsystem 102 comprises a health data management engine 104 (engine 104), a processor 106, and a memory 108. In various embodiments of the present invention, the engine 104 has multiple units which work in conjunction with each other for personalized health data management. The various units of the engine 104 are operated via the processor 106. The processor 106 is a specific-purpose processor which is specifically programmed to execute instructions stored in the memory 108 for executing respective functionalities of the units of the engine 104 in accordance with various embodiments of the present invention.

In another embodiment of the present invention, the subsystem 102 may be implemented in a cloud computing architecture in which data, applications, services, and other resources are stored and delivered through shared datacenters. In an exemplary embodiment of the present invention, certain functionalities of the subsystem 102 are delivered to a user as Software as a Service (SaaS) or a Platform as a Service (PaaS) over a communication network.

In another embodiment of the present invention, the subsystem 102 may be implemented in a client-server architecture. In this embodiment of the present invention, a client terminal accesses a server hosting the subsystem 102 over a communication network. The client terminals may include but are not limited to a smart phone, a computer, a tablet, microcomputer or any other wired or wireless terminal. The server may be a centralized or a decentralized server.

In an embodiment of the present invention, the engine 104 comprises a data collection and pre-processing unit 112, a data analysis unit 114, a feature extraction unit 116, a feature augmentation unit 118 and an insights and recommendations generation unit 120.

In operation, in an embodiment of the present invention, the data collection and pre-processing unit 112 receives and collects one or more data types from the data source unit 110. The data source unit 110 may be connected to multiple data sources from which the data is collected. The data sources may include, but are not limited to, databases, health monitoring devices (e.g., smartwatch, and wearable devices such as, Apple® healthkit, Google® fit, fitbit, etc.), integrated additional health data sources and Electronic Health Records (EHR). In an exemplary embodiment of the present invention, the data source unit 110 may be installed as an application in a user device, which collects data from the health monitoring devices, from the databases or from a portal and transmits the collected data to the data collection and pre-processing unit 112. The data types collected may include biometric data such as, but are not limited to, health reports, blood reports from blood monitoring machines, data from health monitoring devices, X-ray reports, medical test reports, prescriptions, doctor recommendations for critical illness and improvements, genetic information and effect of environmental factors. The collected data is present in different formats such as word format, PDF format, digital format, etc.

In various embodiments of the present invention, the data collection and pre-processing unit 112 pre-processes the collected data type. In an embodiment of the present invention, the data collection and pre-processing unit 112 cleans the collected data types by using data cleaning techniques such as pandas®, Numpy®, etc. Further, the data collection and pre-processing unit 112 removes any irrelevant data from the collected data types, corrects misspellings, and standardizes the text format of the collected data type. The data collection and pre-processing unit 112 breaks down the text present in the collected data types into individual tokens (e.g., words or phrases) for easier analysis. The data collection and pre-processing unit 112 further converts the text present in the collected data to a standard format (e.g., lowercasing and removing punctuation from the collected data). Further, the data collection and pre-processing unit 112 converts the collected data types which may be present in raw form into a structured format suitable for analysis. The collected data types are converted to a structured format by the data collection and pre-processing unit 112 by aggregating data over specific time intervals or implementing data conversion techniques.

In an embodiment of the present invention, the data collection and pre-processing unit 112 performs one or more data extraction operations on the pre-processed collected data types to generate processed collected data types. The data collection and pre-processing unit 112 performs one or more Optical Character Recognition (OCR) techniques to convert the pre-processed collected data types into a machine-readable format. For example, data types present in PDFs format are converted into a machine-readable format. The OCR techniques may include, but are not limited to, tesseract library and Google cloud vision. Further, the data collection and pre-processing unit 112 implements the data extraction operations by employing one or more Natural Language Processing (NLP) techniques to parse and structure the pre-processed collected data types subsequent to implementation of the data extraction operation. Using NLP techniques, one or more key entities such as patient names, dates, medical terms, and other relevant information are identified in the received data types. The NLP techniques include, but are not limited to, spaCy® and Natural Language Toolkit (NLTK). Further, the data collection and pre-processing unit 112 collects data from health monitoring devices by using Application Programming Interfaces (APIs) and Software Development Kits (SDKs). The APIs and SDKs allow access to various health metrics such as heart rate, footsteps, sleep patterns, etc. Further, the data collection and pre-processing unit 112 implements the one or more data extraction operations by employing a real-time data streaming technique to continuously collect data from health monitoring devices.

In various embodiments of the present invention, the data analysis unit 114 receives the processed data type from the data collection and pre-processing unit 112 for analysis. In an embodiment of the present invention, the data analysis unit 114 generates a sequence of one or more intelligent prompts which are employed to detect abnormalities and deviations by analyzing the processed data types. In an embodiment of the present invention, the sequence of intelligently generated prompts is provided by the data analysis unit 114 to Artificial Intelligence (AI) models to determine abnormalities and deviations in the data types. In an example, abnormalities and deviations are detected in key health parameters present in the data types such as blood components, blood pressure, heartbeat, sugar levels (A1C), etc. In an embodiment of the present invention, the data analysis unit 114 generates warnings for abnormal health parameters associated with the data types that may require immediate support or guidance.

In an embodiment of the present invention, the feature extraction unit 116 receives the analyzed data types from the data analysis unit 114 for extracts one or more health features data from the analyzed data types by applying one or more feature extraction techniques. The feature extraction unit 116 applies the feature extraction techniques by identifying and classifying entities in the analyzed data types. The entities may include diseases, medications, symptoms, etc. In an exemplary embodiment of the present invention, a Named Entity Recognition (NER) technique is implemented by the feature extraction unit 116 for identifying and classifying entities in the analyzed data. Further, the feature extraction unit 116 converts text present in the analyzed data types into numerical representations such as embeddings. The embeddings capture the semantic meaning of the text, and the embeddings are used for further analysis of the collected data types. The feature extraction unit 116 uses models such as Bidirectional Encoder Representations from Transformers (BERT), Generative Pre-trained Transformer (GPT), etc. for converting text into embeddings. In an embodiment of the present invention, the feature extraction unit 116 extracts features data from time-series data associated with the analyzed data types. The time series data include, but are not limited to, average heart rate, step count trends, and sleep duration patterns. In an embodiment of the present invention, the feature extraction unit 116 further computes statistical features data of the analyzed data types for summarizing the data types. The statistical features are computed using one or more statistical computation techniques such as mean, median, standard deviation, and variance.

In an embodiment of the present invention, the feature augmentation unit 118 receives the extracted health features data from the feature extraction unit 116. The feature augmentation unit 118 employs one or more Machine Learning (ML) models to augment the extracted health features data. The feature augmentation unit 118 employs a classification model to classify the extracted health features data into one or more pre-defined categories. The pre-defined categories include, but are not limited to, diagnosis, treatment, and patient history. The classification models include, but are not limited to, logistic regression, and Support Vector Machine (SVM). Further, the feature augmentation unit 118 employs a clustering model to group similar data points associated with the extracted health features data together by using clustering techniques for identifying patterns and trends in the health data associated with the extracted features data. The clustering techniques include, but are not limited to, K-means clustering and hierarchical clustering. Further, the feature augmentation unit 118 employs a predictive model to predict health outcomes based on the extracted health features data. In an example, predicted health outcomes include predicting the likelihood of a person developing a certain illness based on medical history. Further, the feature augmentation unit 118 employs the ML models for analyzing the extracted features data in order to identify anomalies and patterns in the health features data. In an exemplary embodiment of the present invention, the feature augmentation unit 118 employs deep learning models such as Long Short-Term Memory (LSTM) for complex time-series analysis for the extracted features data.

In an embodiment of the present invention, the insights and recommendations generation unit 120 receives and processes the analyzed health features data for generating insights and recommendations with respect to the health of the user. The insights and recommendations generation unit 120 employs GenAI agents for generating summaries of the extracted health feature data associated with the health data. Generation of the summary aids in providing a quick overview of a patient's medical history or treatment plan. Further, the insights and recommendations generation unit 120 identifies anomalies or outliers in the health features data that may indicate potential health issues. The insights and recommendations generation unit 120 uses outcome of the predictive models to compute one or more future health metrics based on historical data. For example, predicting the likelihood of a user meeting his/her fitness goals. The insights and recommendations provide one or more predictive actions to identify expected health challenges for guiding the user on health improvement and also provides the users health information to healthcare service providers (e.g., doctors, pharmacy, etc.) and support functions (e.g., insurance providers, financial organizations, etc.) for enabling them to forecast user health parameters.

In an embodiment of the present invention, the insights and recommendations generation unit 120 generates personalized health insights and recommendations for the user based on analyzed health features data. For example, personalized health insights and recommendations includes suggesting changes in activity levels, changes in sleep habits, lifestyle changes, change in medications which needs to be approved by doctors, health counselling services performed by specialists, information shared with financial and insurance organization which are linked to the health parameters, etc. Further, the recommendations may relate to recommending medicines, food items, receipes, supplements, physical activities and lifestyle changes to the user. The insights and recommendations generation unit 120 proactively monitors and tracks the users' health data and alerts the user with immediate remediation actions. For example, if it is identified that blood pressure of the user is shooting up, then the user is alerted to act on the recommendations such as resting, performing some basic doctor recommended measures, etc. The insights and recommendations generated by the insights and recommendations generation unit 120 are rendered via the output unit 122 via a User Interface (UI) for visualization.

In an embodiment of the present invention, the insights and recommendations generation unit 120 is configured to automatically trigger rendering of the one or more action items via the output unit 122 based on the generated insights and recommendations. In an embodiment of the present invention, a first action item relates to the insights and recommendations generation unit 120 communicating with one or more emergency services based on the generated insights and recommendations. The emergency services include but are not limited to, ambulance, hospital emergency sections for admissions, on-duty specialist availability, enabling support from the patient regular consulting doctor, insurance approvals for treatments or any other support which the patient might need in case of emergencies or non-emergencies.

In another embodiment of the present invention, a second action item relates to the insights and recommendations generation unit 120 communicating with one or more collaborative services for consumption based on the user's choice of subscription. The collaborative services include, but are not limited to, a pharmacy, a retail store, a food supplier, insurance service, financial services, wellness centres, etc. In an example, if the user subscribes to an order management by a food supplier, then the user is provided with details of the food that is required to be delivered to the user based on the generated insights and recommendations. In another example, if the user subscribes to a wellness centre with a trainer, then the information of the user is provided to a trainer at the wellness trainer to provide exercise routines. In another example, if the user subscribes to a pharmacy, then one or more medicines are recommended based on doctor approval, and the pharmacy may deliver the medicines to the user. In another example, if user requires a doctor's consultation, then the insights and recommendations generation unit 120 interacts with the general physician and books an appointment with the report insights published to the doctor.

In yet another embodiment of the present invention, a third action item relates to the insights and recommendations generation unit 120 communicating with other applications or internet applications for fetching regular feeds into the subsystem 102 to render a consolidated view of the health parameters. For example, the applications may include mobile applications or internet applications including, but are not limited to, a wellness application, body health parameter application, A1C blood sugar level, an external public website which can share information about disease spreads, etc.

In various exemplary embodiments of the present invention, the subsystem 102 is developed using a variety of programming languages and frameworks to ensure robust and efficient development across different platforms. In an exemplary embodiment of the present invention, Python® programming language is extensively used for AI and machine learning due to its comprehensive libraries and frameworks. Frontend is developed by using JavaScript® and TypeScript®, particularly with frameworks such as React Native®. Native iOS and Android development are facilitated using Swift® and Kotlin®, respectively. The subsystem 102 includes several AI and machine learning frameworks to build and train sophisticated models. TensorFlow® an open-source platform, is utilized for machine learning tasks. PyTorch® is another preferred library known for its flexibility and ease of use. Hugging Face® Transformers are employed for implementing the NLP models.

In various exemplary embodiments of the present invention, the subsystem 102 performs text processing and analysis by using spaCy®. Additionally, the NLTK provides tools for working with human language data for enhancing capabilities in NLP. Integration with external data sources, wearables, and other health systems is carried out by using RESTful® APIs. For more efficient data querying, GraphQL® is utilized. Further, Fast Healthcare Interoperability Resources (FHIR) standard is implemented for exchanging healthcare information electronically. The invention leverages cloud platforms such as AWS®, Google Cloud Platform (GCP), and Azure® for AI, machine learning, data storage, and processing services. Google Cloud AI provides pre-trained models and tools for building custom AI solutions, while Azure Cognitive Services offers APIs for vision, speech, language, and decision-making.

In various exemplary embodiments of the present invention, in the subsystem 102, structured data storage is carried out by using SQL databases such as PostgreSQL® and MySQL®. Also, NoSQL databases such as MongoDB® and Firebase® are employed for unstructured data, ensuring flexible and scalable data management. Further, React Native® is used for building cross-platform mobile applications using JavaScript®. Flutter®, a UI toolkit, is employed for creating natively compiled applications for mobile from a single codebase, enhancing mobile development capabilities. Further, secure authentication and authorization is ensued by using OAuth 2.0®. Compliance with healthcare data protection regulations such as Health Insurance Portability and Accountability Act (HIPAA) and General Data Protection Regulation (GDPR) is maintained to protect sensitive information. Further, for ensuring consistency across environments, Docker® is used for containerizing applications. Kubernetes® orchestrates these containerized applications, while Jenkins® and GitHub® Actions facilitate continuous integration and continuous deployment (CI/CD).

In various exemplary embodiments of the present invention, the subsystem 102 uses APIs and SDKs to integrate with wearables, EHR systems, and other data sources, enhancing interoperability and data collection capabilities. The workflow for GenAI integration involves several steps. Data is collected using APIs from wearables, EHR systems, and other sources. This data is then cleaned and preprocessed using Python libraries like pandas® and NumPy®. AI models are trained using TensorFlow or PyTorch and subsequently deployed on cloud platforms like AWS or GCP. The trained models are integrated with a mobile application using RESTful APIs or GraphQL®. The application's frontend is developed using React Native® or Flutter®, and OAuth 2.0 is implemented for secure access, thereby ensuring compliance with healthcare regulations.

In various exemplary embodiments of the present invention, in the subsystem 102, data integration is achieved through various methods. Libraries are implemented to read and extract data from documents and PDFs. SDKs from wearable manufacturers such as Apple HealthKit® and Google Fit®, are used to collect data. Web scraping tools or APIs gather data from websites and other apps. UI and user experience (UX) design process involves creating wireframes and prototypes to visualize the application, thereby ensuring it is intuitive and easy to navigate. Frontend development is carried out using frameworks like React Native® or Flutter® for cross-platform compatibility, while backend development focuses on server-side logic, database management, and API integrations.

In various exemplary embodiments of the present invention, in the subsystem 102, unit testing is conducted to test individual components for functionality. Integration testing ensures all integrated systems work seamlessly together. User acceptance testing (UAT) is performed with users to gather feedback and make necessary adjustments. Continuous monitoring and maintenance are carried out to address performance issues and provide regular updates. Security and privacy measures include implementing encryption for data storage and transmission, ensuring access control so only authorized users can access sensitive data, and conducting regular security audits to identify and fix vulnerabilities. Post-launch support involves collecting and analyzing user feedback to improve the application and regularly updating it with new features and enhancements.

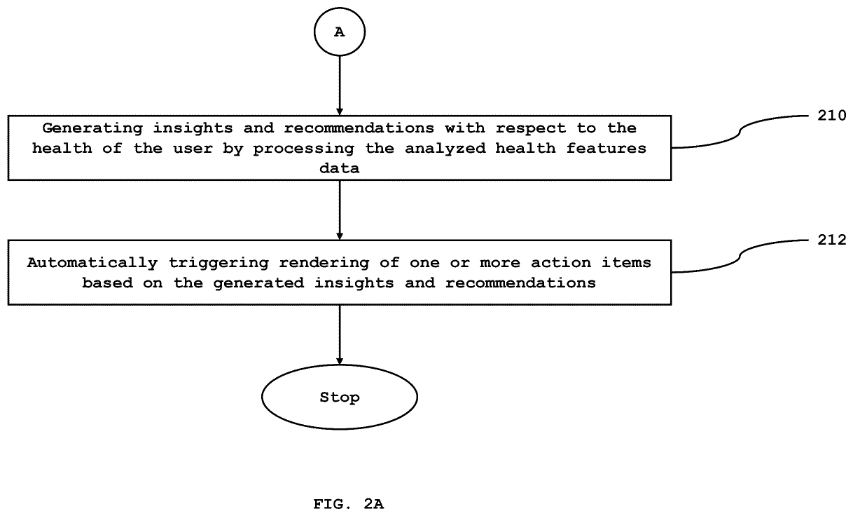

FIG. 2 and FIG. 2A illustrate a flowchart depicting an AI based method for personalized health data management, in accordance with various embodiments of the present invention.

At step 202, data extraction operations are performed on collected data types to obtain processed data types. In an embodiment of the present invention, one or more data types are collected from various data sources. The data sources include, but are not limited to, databases, health monitoring devices (e.g., smartwatch, and wearable devices such as Apple® healthkit, Google® fit, fitbit, etc.), integrated additional health data sources and EHR. The data types may include, but are not limited to, health reports, blood reports from blood monitoring machines, data from health monitoring devices, X-ray reports, medical test reports, prescriptions, doctor recommendations for critical illness and improvements, genetic information and effect of environmental factors. The collected data is present in different formats such as, word format, PDF format, digital format, etc.

In various embodiments of the present invention, the collected data types are pre-processed using one or more data processing operations. In an embodiment of the present invention, the data processing operations include cleaning collected data types by using data cleaning techniques such as pandas®, Numpy®, etc. Further, the data processing operations include removing any irrelevant data from the collected data types, misspellings are corrected, and the text format of the collected data type is standardized. Further, the text present in the collected data types is broken down into individual tokens (e.g., words or phrases) for easier analysis. The text present in the collected data is converted to a standard format (e.g., lowercasing and removing punctuation from the collected data). Further, the collected data type which may be present in raw form is converted into a structured format suitable for analysis. The collected data types are converted to a structured format by aggregating data over specific time intervals or implementing data conversion techniques.

In an embodiment of the present invention, one or more data extraction operations are performed on the pre-processed collected data types to obtain processed data types. Firstly, OCR techniques are performed to convert the pre-processed collected data types into a machine-readable format. For example, data types present in PDFs format are converted into a machine readable format. The OCR techniques include, but are not limited to, tesseract library and Google cloud vision. Further, the one or more data extraction operations are implemented by employing one or more NLP techniques to parse and structure the collected data types subsequent to implementation of the data extraction operation. The implementation of NLP techniques identifies one or more key entities such as patient names, dates, medical terms, and other relevant information in the received data types. The NLP techniques implemented include, but are not limited to, spaCy® and NLTK. Further, data from health monitoring devices is collected by using APIs and SDKs. The APIs and SDKs allow access to various health metrics such as heart rate, foot-steps, sleep patterns, etc. Further, the one or more data extraction operations are implemented by employing a real-time data streaming technique to continuously collect data from health monitoring devices.

At step 204, the processed data types are analyzed for detecting abnormalities and deviations. In an embodiment of the present invention, abnormalities and deviations are detected in key health parameters present in the data types such as blood components, blood pressure, heartbeat, sugar levels (A1C), etc. In an embodiment of the present invention, warnings are generated for abnormal health parameters associated with the data types that may require immediate support or guidance.

At step 206, one or more health features data are extracted from the analyzed data types by applying one or more feature extraction techniques. In an embodiment of the present invention, the one or more feature extraction techniques are applied by identifying and classifying entities in the analyzed data types. The entities may include diseases, medications, symptoms, etc. In an exemplary embodiment of the present invention, a NER technique is implemented for identifying and classifying entities in the analyzed data. Further, text present in the analyzed data types is converted into numerical representations such as embeddings. The embeddings capture the semantic meaning of the text and the embeddings are used for further analysis of the collected data types. Further, models such as BERT, GPT, etc. are used for converting text into embeddings. In an embodiment of the present invention, features data is extracted from time-series data associated with the analyzed data types. The time series data includes, but is not limited to, average heart rate, step count trends, and sleep duration patterns. In an embodiment of the present invention, statistical features data of the analyzed data types is computed for summarizing the data types. The statistical features are computed using one or more statistical computation techniques such as mean, median, standard deviation, and variance.

At step 208, one or more ML models are employed to augment the extracted health features data. In an embodiment of the present invention, firstly, a classification model is employed to classify the extracted health features data into one or more pre-defined categories. The pre-defined categories include, but are not limited to, diagnosis, treatment, and patient history. The classification models include, but are not limited to, logistic regression, and SVM. Further, a clustering model is employed to group similar data points associated with the extracted health features data together by using clustering techniques for identifying patterns and trends in the health data associated with the extracted features data. The clustering techniques include, but are not limited to, K-means clustering and hierarchical clustering. Further, a predictive model is employed to predict health outcomes based on the extracted health features data. In an example, predicted health outcomes include predicting the likelihood of a person developing a certain illness based on medical history. Further, the generated ML models are employed for analyzing the extracted features data in order to identify anomalies and patterns in the health features data. Further, deep learning models such as LSTM are implemented for complex time-series analysis for the extracted features data.

At step 210, insights and recommendations are generated with respect to the health of the user by processing the analyzed health features data. In an embodiment of the present invention, the analyzed health features data is processed for generating insights and recommendations with respect to the health of the user. GenAI agents are employed for generating summaries of the extracted health feature data associated with the health data. Further, anomalies or outliers in the health features data are identified that may indicate potential health issues. Further, outcome of the predictive models is used to compute one or more future health metrics based on historical data. For example, outcome of the predictive models includes predicting likelihood of a user meeting his/her fitness goals. The insights and recommendations provide one or more predictive actions to identify expected health challenges for guiding the user regarding health improvement and also provides the users health information to healthcare service providers (e.g., doctors, pharmacy, etc.) and support functions (e.g., insurance providers, financial organizations, etc.) for enabling them to forecast user health parameters.

In an embodiment of the present invention, personalized health insights and recommendations are generated for the user based on analyzed health features data. For example, personalized health insights and recommendations include suggesting changes in activity levels, changes in sleep habits, lifestyle changes, change in medications which needs to be approved by doctors, health counselling services performed by specialist, information shared with financial and insurance organization which are linked to the health parameters, etc. Further, the recommendations may relate to recommending medicines, food items, receipes, supplements, physical activities and lifestyle changes to the user. Further, the users' health data and alerts are proactively monitored and tracked with immediate remediation actions. For example, if it is identified that blood pressure of the user is shooting up, then the user is alerted to act on the recommendations such as, resting, performing some basic doctor recommended measures, etc. The generated insights and recommendations generation are rendered for visualization.

At step 212, one or more action items are automatically triggered based on the generated insights and recommendations. In an embodiment of the present invention, a first action item relates to communicating with one or more emergency services based on the generated insights and recommendations. The emergency services include, but are not limited to, ambulance, hospital emergency sections for admissions, on-duty specialist availability, enabling support from the patient regular consulting doctor, insurance approvals for treatments or any other support which the patient might need in case of emergencies or non-emergencies.

In another embodiment of the present invention, a second action item relates to communicating with one or more collaborative services based on the insights and recommendations rendered for consumption based on the user's choice of subscription. The collaborative services include, but are not limited to, a pharmacy, a retail store, a food supplier, insurance service, financial services, wellness centres, etc. In an example, if the user subscribes to an order management by a food supplier, then the user is provided with details of the food that is required to be delivered to the user based on the generated insights and recommendations. In another example, if the user subscribes to a wellness centre with a trainer, then the information of the user is provided to a trainer at the wellness trainer to provide exercise routines. In another example, if the user subscribes to a pharmacy, then one or more medicines are recommended based on doctor approval, and the pharmacy may deliver the medicines to the user. In yet another example, if user requires a doctor's consultation, then output interacts with the general physician and book an appointment with the report insights published to the doctor.

In yet another embodiment of the present invention, a third action item relates to communicating with other applications or internet applications for fetching regular feeds to render a consolidated view of the health parameters. For example, applications include mobile applications or internet applications including, but are not limited to, a wellness application, body health parameter application, A1C monitor, an external public website which can share information about the disease spreads, etc.

Advantageously, in accordance with various embodiments of the present invention, the present invention provides for Gen AI based personalized health data management in an automated manner. Examples of the present invention provide for effectively processing large volume of health data for generating healthcare related insights and recommendations. Examples of the present invention provide for eliminating or reducing manual intervention in health data management for fast and accurate health data management. Further, Examples of the present invention provide for real-time adaptation of health management systems to changing health needs and preferences. Furthermore, examples of the present invention provide for efficiently integrating data from various sources for comprehensive health management. Furthermore, present invention provides for proactively engaging with finance and insurance organizations for enhancing experience for patients and customers. Also, examples of the present invention provide for enhanced health outcomes and promote proactive healthcare management by integrating state-of-the-art technologies and data-driven insights. Yet further, the example of the present invention provides for effectively processing large volume of health data for generating user specific healthcare related insights and recommendations.

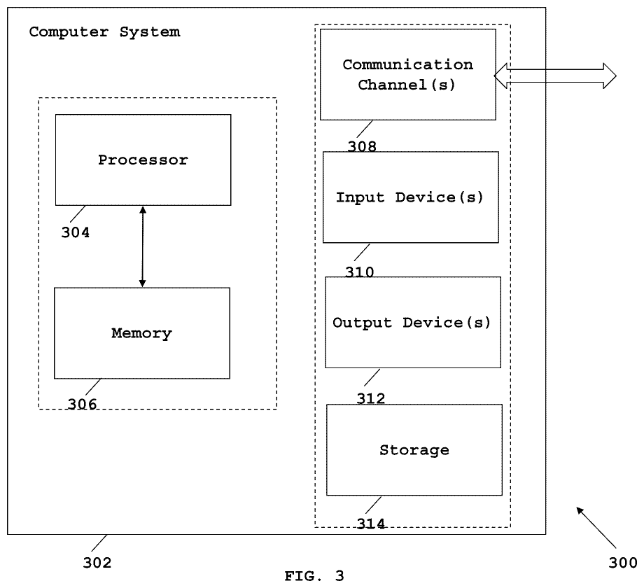

FIG. 3 illustrates an exemplary computer system in which various embodiments of the present invention may be implemented. The computer system 302 comprises a specific-purpose processor 304 and a memory 306. The processor 304 executes program instructions and is a real processor. The computer system 302 is not intended to suggest any limitation as to scope of use or functionality of described embodiments. For example, the computer system 302 may include, but not limited to, a programmed microprocessor, a micro-controller, a peripheral integrated circuit element, and other devices or arrangements of devices that are capable of implementing the steps that constitute the method of the present invention. In an embodiment of the present invention, the memory 306 may store software for implementing various embodiments of the present invention. The computer system 302 may have additional components. For example, the computer system 302 includes one or more communication channels 308, one or more input devices 310, one or more output devices 312, and storage 314. An interconnection mechanism (not shown) such as a bus, controller, or network, interconnects the components of the computer system 302. In various embodiments of the present invention, operating system software (not shown) provides an operating environment for various softwares executing in the computer system 302 and manages different functionalities of the components of the computer system 302.

The communication channel(s) 308 allow communication over a communication medium to various other computing entities. The communication medium provides information such as program instructions, or other data in a communication media. The communication media includes, but not limited to, wired or wireless methodologies implemented with an electrical, optical, RF, infrared, acoustic, microwave, Bluetooth, or other transmission media.

The input device(s) 310 may include, but not limited to, a keyboard, mouse, pen, joystick, trackball, a voice device, a scanning device, touch screen or any another device that is capable of providing input to the computer system 302. In an embodiment of the present invention, the input device(s) 310 may be a sound card or similar device that accepts audio input in analog or digital form. The output device(s) 312 may include, but not limited to, a user interface on CRT or LCD, printer, speaker, CD/DVD writer, or any other device that provides output from the computer system 302.

The storage 314 may include, but not limited to, magnetic disks, magnetic tapes, CD-ROMs, CD-RWs, DVDs, flash drives or any other medium which can be used to store information and can be accessed by the computer system 302. In various embodiments of the present invention, the storage 314 contains program instructions for implementing the described embodiments.

Examples of the present invention may suitably be embodied as a computer program product for use with the computer system 302. The method described herein is typically implemented as a computer program product, comprising a set of program instructions which is executed by the computer system 302 or any other similar device. The set of program instructions may be a series of computer readable codes stored on a tangible medium, such as a computer readable storage medium (storage 314), for example, diskette, CD-ROM, ROM, flash drives or hard disk, or transmittable to the computer system 302, via a modem or other interface device, over either a tangible medium, including but not limited to optical or analogue communications channel(s) 308. The implementation of the invention as a computer program product may be in an intangible form using wireless techniques, including but not limited to microwave, infrared, Bluetooth, or other transmission techniques. These instructions can be preloaded into a system or recorded on a storage medium such as a CD-ROM or made available for downloading over a network such as the internet or a mobile telephone network. The series of computer readable instructions may embody all or part of the functionality previously described herein.

Examples of the present invention may be implemented in numerous ways including as a system, a method, or a computer program product such as a computer readable storage medium or a computer network wherein programming instructions are communicated from a remote location.

While the exemplary embodiments of the present invention are described and illustrated herein, it will be appreciated that they are merely illustrative. It will be understood by those skilled in the art that various modifications in form and detail may be made therein without departing from or offending the scope of the invention.

We claim:

1. An Artificial Intelligence (AI) based system for personalized health data management, the system comprises:
a memory storing program instructions;
a processor executing instructions stored in the memory; and
a health data management engine executed by the processor and configured to:
perform one or more data extraction operations on one or more pre-processed data types to obtain processed data types, wherein the data types are collected from multiple data sources;
analyze the processed data types for detecting abnormalities and deviations in the collected data types by providing a sequence of prompts to AI models;
extract one or more health features data from the analyzed data types by using one or more feature extraction techniques;
employ one or more Machine Learning (ML) models to augment the extracted health features data in order to identify anomalies and patterns in the health features data; and
generate insights and recommendations associated with health of a user based on processing of the analyzed features data, wherein one or more action items are triggered based on the generated insights and recommendations.

2. The system as claimed in claim 1, wherein a data collection and pre-processing unit pre-processes the collected data type by cleaning the collected data types using data cleaning techniques, removing irrelevant data from the collected data types, correcting misspellings, and standardizing text format of the collected data, and wherein the text present in the collected data types is broken down into individual tokens for easier analysis, and wherein the collected data types are converted to a structured format by the data collection and pre-processing unit by aggregating data over specific time intervals or implementing data conversion techniques.

3. The system as claimed in claim 1, wherein the health data management engine comprises a data collection and pre-processing unit executed by the processor and is configured to perform the data extraction operations on the collected data types comprising one or more Optical Character Recognition (OCR) techniques to convert collected data types into a machine-readable format, and wherein one or more Natural Language Processing (NLP) techniques are employed to parse and structure the collected data types subsequent to implementation of the data extraction operation, and wherein a real-time data streaming technique is employed to continuously collect data from one or more health monitoring devices.

4. The system as claimed in claim 1, wherein the health data management engine comprises a data analysis unit executed by the processor and is configured to generate the sequence of intelligent prompts which are provided to the AI models to detect abnormalities and deviations in the collected data types and generate warnings for one or more abnormal health parameters associated with the data types.

5. The system as claimed in claim 1, wherein the health data management engine comprises a feature extraction unit executed by the processor and is configured to extract one or more health features data from the analyzed data types by applying the one or more feature extraction techniques to identify and classify entities in the analyzed data types based on a Named Entity Recognition (NER) technique.

6. The system as claimed in claim 5, wherein the feature extraction unit converts text present in the analyzed data types into numerical representations referred to as embeddings, the embeddings capture the semantic meaning of the text present in the collected data types and are used for analysis of the collected data types, and wherein the feature extraction unit extracts features data from time-series data associated with the analyzed data types, the time series data comprises average heart rate, step count trends, and sleep duration patterns, and wherein the feature extraction unit computes statistical features data of the analyzed data types for summarizing the data types.

7. The system as claimed in claim 1, wherein the health data management engine comprises a feature augmentation unit executed by the processor and is configured to employ a classification model to classify the extracted health features data into one or more pre-defined categories, the pre-defined categories comprise diagnosis, treatment, and patient history, and wherein a clustering model is employed to group similar data points associated with the extracted features data together by using clustering techniques for identifying patterns and trends in the health data associated with the extracted features data, and wherein a predictive model is employed to predict health outcomes based on the extracted features data, and wherein outcome of the predictive models is used to compute one or more future health metrics based on historical data.

8. The system as claimed in claim 1, wherein the health data management engine comprises an insights and recommendations generation unit executed by the processor and is configured to employ one or more GenAI models for generating summaries of the extracted health feature data associated with the health data, and wherein the insights and recommendations generation unit identifies anomalies and outliers in the features data that indicate user's potential health issues.

9. The system as claimed in claim 1, wherein the insights and recommendations generation unit provide one or more predictive actions for identifying expected health challenges to guide the user on health improvement and provides user's health information to healthcare service providers and support functions for enabling them to forecast user's health parameters.

10. The system as claimed in claim 8, wherein the insights and recommendations generation unit proactively monitor and tracks the users' health data and alerts the user with immediate remediation actions.

11. The system as claimed in claim 1, wherein the insights and recommendations generation unit provides one or more predictive actions including a first action item relating to communicating with one or more emergency services based on the generated insights and recommendations, a second action item relating to communicating with one or more collaborative services based on the insights and recommendations rendered for consumption based on the user's choice of subscription, and a third action item relating to communicating with other applications or internet applications for fetching regular feeds to render a consolidated view of the health parameters.

12. An AI based method for personalized health data management, the method is implemented by a processor executing instructions stored in a memory, the method comprising:
performing one or more data extraction operations on one or more data types to obtain processed data types, wherein the data types are collected from multiple data sources;
analyzing the data types for detecting abnormalities and deviations in the collected data types by providing a sequence of prompts to AI models;
extracting one or more health features data from the analyzed data types by using one or more feature extraction techniques;
employing one or more ML models to augment the extracted health features data in order to identify anomalies and patterns in the health features data; and
generating insights and recommendations associated with health of a user based on processing of the analyzed health features data, wherein one or more action items are triggered based on the generated insights and recommendations.

13. The method as claimed in claim 12, wherein the step of performing one or more data extraction operations comprises converting collected data types into a machine-readable format using one or more OCR techniques, and parsing and structuring the collected data types subsequent to implementation of the data extraction operation using one or more NLP techniques, and wherein a real-time data streaming technique is employed to continuously collect data from one or more health monitoring devices.

14. The method as claimed in claim 13, wherein collected data type are pre-processed by cleaning the collected data types by using data cleaning techniques, removing irrelevant data from the collected data types, correcting misspellings, and standardizing text format of the collected data, and wherein text present in the collected data types is broken down into individual tokens for easier analysis, and wherein the collected data types are converted to a structured format by aggregating data over specific time intervals or implementing data conversion techniques.

15. The method as claimed in claim 13, wherein the step of performing one or more data extraction operations comprises extracting one or more health features data from the analyzed data types by identifying and classifying entities in the analyzed data types based on a Named Entity Recognition (NER) technique.

16. The method as claimed in claim 15, wherein the step of extracting one or more health features data from the analyzed data types comprises converting text present in the analyzed data types into numerical representations referred to as embeddings, the embeddings capture the semantic meaning of the text present in the collected data types and the embeddings are used for analysis of the collected data types, and wherein features data is extracted from time-series data associated with the analyzed data types, the time series data comprises average heart rate, step count trends, and sleep duration patterns, and wherein statistical features data of the analyzed data types is computed for summarizing the data types.

17. The method as claimed in claim 13, wherein the step of employing one or more ML models comprises employing a classification model to classify the extracted health features data into one or more pre-defined categories, the pre-defined categories comprises diagnosis, treatment, and patient history, employing a clustering model to group similar data points associated with the extracted features data together by using clustering techniques for identifying patterns and trends in the health data associated with the extracted features data, and employing a predictive model to predict health outcomes based on the extracted features data, and wherein outcome of the predictive models is used to compute one or more future health metrics based on historical data.

18. The method as claimed in claim 13, wherein the step of generating insights and recommendations comprises employing one or more GenAI models for generating summaries of the extracted health feature data associated with the health data, and wherein anomalies and outliers are identified in the features data that indicate user's potential health issues.

19. The method as claimed in claim 12, wherein the one or more action items comprises a first action item relating to communicating with one or more emergency services based on the generated insights and recommendations, a second action item relating to communicating with one or more collaborative services based on the insights and recommendations rendered for consumption based on the user's choice of subscription, and a third action item relating to communicating with other applications or internet applications for fetching regular feeds to render a consolidated view of the health parameters.

20. A computer program product comprising:
a non-transitory computer-readable medium having computer-readable program code stored thereon, the computer-readable program code comprising instructions that, when executed by a processor, cause the processor to:
perform one or more data extraction operations on one or more data types to obtain processed data types, wherein the data types are collected from multiple data sources;
analyze the processed data types for detecting abnormalities and deviations in the collected data types by providing a sequence of prompts to AI models;
extract one or more health features data from the analyzed data types by using one or more feature extraction techniques;
employ one or more Machine Learning (ML) models to augment the extracted health features data to identify anomalies and patterns in the health features data; and
generate insights and recommendations associated with health of a user based on processing of the analyzed features data, wherein one or more action items are triggered based on the generated insights and recommendations.
